(19) United States
(12) Patent Application Publication
(10) Pub. No.: US 2016/0324689 A1
(43) Pub. Date: Nov. 10, 2016

(54) Duct For Tear Flow

(71) Applicant: 3D Global Biotech Inc., Taipei City (TW)

(72) Inventors: Yun-Dun Shen, New Taipei City (TW); Keng-Liang Ou, Taipei City (TW)

(73) Assignee: 3D GLOBAL BIOTECH INC., Taipei City (TW)

(21) Appl. No.: 14/705,968

(22) Filed: May 7, 2015

(51) Int. Cl. A61F 9/007; A61M 27/00

(52) U.S. Cl. CPC A61F 9/00772; A61M 27/002; A61M 2210/0618; A61M 2205/0216

(57) ABSTRACT

A duct for tear flow, for connecting the nasal cavity and the lacrimal sac, comprises: a tube, which is set across the nasal mucosa, nasal bone wall and lacrimal sac mucosa, so that the nasal cavity and the lacrimal sac are connected; a positioning element, set on the front of the tube, and fastened on the lacrimal sac mucosa; so that tears flow into the nasal cavity due to a connection between the nasal cavity and the lacrimal sac.

### FIELD OF THE INVENTION

The present invention relates to a duct for tear flow; particularly to a duct for tear flow which is set across a nasal mucosa, a nasal bone wall and a lacrimal sac mucosa, so that the nasal cavity and the lacrimal sac are connected; so that tears in the lacrimal sac can flow into the nasal cavity.

### BACKGROUND OF THE INVENTION

Tears are secreted by the lacrimal gland, mainly for moisturizing the surface of the eyeballs. Superfluous tears will flow into the lacrimal sac, then through the nasolacrimal duct into the nasal cavity.

As a rule, the lacrimal gland secretes tears all the time, but the nasolacrimal duct occasionally is obstructed. There are many reasons for obstruction in the nasolacrimal duct, e.g. inflammation and bacterial infection due to improper remove of makeup around the eyes.

Besides cleaning an obstructed nasolacrimal duct, surgery to connect nasal cavity and lacrimal sac is an option. Process of the surgery is as follows: setting a passage, which crosses through the nasal mucosa, nasal bone wall and lacrimal sac mucosa, then taking flap surgery, wherein tissue around the passage will not close the passage after healing. Therefore, even if the nasolacrimal duct is obstructed, tears in the lacrimal sac can still flow into the nasal cavity.

However, skill for such a surgery is very demanding. The steps are sophisticated and time consuming, particularly in the steps of the flap surgery, if only a slightest mistake happens, the passage could be obstructed by tissue around the passage.

In view of these disadvantages the inventor tried the continuous testing and improvement and developed the present invention.

### SUMMARY OF THE INVENTION

The main object of the present invention is to provide a duct for tear flow, wherein nasal cavity and lacrimal sac are connected, so that tears in the lacrimal sac can flow into the nasal cavity.

For achieving above object the present invention comprises: a tube, having a front end and a real end, set across the nasal mucosa, nasal bone wall and lacrimal sac mucosa, so that the nasal cavity and the lacrimal sac are connected; a positioning element, set on the front end of the tube and fastened on the lacrimal sac mucosa.

Preferably, the surface of the front end on the tube is coated with antihistamine. Thereby it is insured, that the tube cannot be obstructed by the lacrimal sac mucosa.

Preferably, the real end of the tube is an anti-proliferative part protruded from the nasal mucosa. Thereby it is insured, that the tube will not be obstructed by the nasal mucosa.

Preferably, the surface of the anti-proliferative part is coated by antihistamine. Thereby it is insured to a greater degree that the real end of the tube cannot be obstructed by the nasal mucosa.

Preferably, the positioning element has at least two fastening parts protruding from the tube on an outer side thereof, so that the fastening parts are fastened on the lacrimal sac mucosa.

Preferably, the two fastening parts are set at a right angle to the tube, wherein the fastening parts are made of elastic material, so that the front end of the tube can easily cross through the nasal mucosa, bone wall and lacrimal sac mucosa, and easily be fastened on the lacrimal sac mucosa.

Preferably, the two fastening parts are parallel to the tube, wherein the fastening parts are bent outward at a right angle to the tube, so that the front end of the tube can easily cross through the nasal mucosa, bone wall and lacrimal sac mucosa, and be easily fastened on the lacrimal sac mucosa.

Preferably, the positioning element is a wafer body made of elastic material. Thereby the antihistamine coating on the front end of the tube is reduced or saved.

Preferably, there is an edge wall on outer edge of the positioning element, wherein a space for filling and growing of lacrimal sac mucosa exists. Thereby the tube is stabilized thereon.

Preferably, on the rear end of the tube there is a flange set outside the nasal mucosa. Thereby it is insured that the rear end of the tube cannot be obstructed by the nasal mucosa.

Other aspects and advantages of the present invention will become apparent from the following detailed description, taken in conjunction with the accompanying drawings, illustrating by way of example the principles of the present invention.

### BRIEF DESCRIPTION OF THE DRAWINGS

FIG. 1 shows an application of the present invention.

FIG. 2 is an enlarged view of part A of FIG. 1.

FIG. 3 is a perspective view of the present invention.

FIG. 4 shows the front end of the tube of the present invention before being inserted into a passage.

FIG. 5 shows the front end of the tube of the present invention after being inserted into, but not yet passing through a passage.

FIG. 6 shows the front end of the tube of the present invention passing through a passage.

FIG. 7 is a perspective view of a positioning element of the present invention as a wafer body.

FIG. 8 is a sectional view of a positioning element of the present invention having an edge wall.

FIG. 9 shows a relevant variant angle between a positioning element and a tube of the present invention.

### DESCRIPTION OF THE PREFERRED EMBODIMENTS

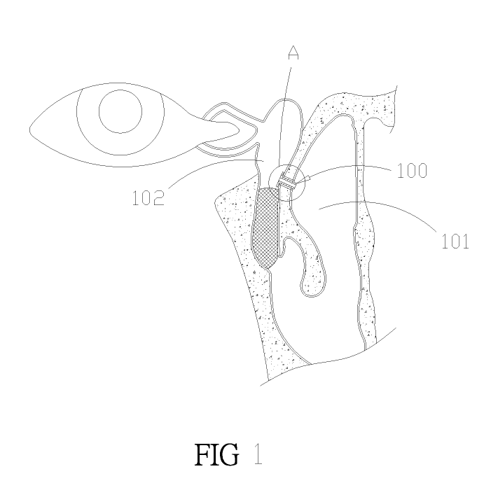

FIG. 1˜6 shows the first embodiment of the present invention. As shown in FIG. 1, a duct 100 for tear flow of the present invention is applied for connecting a nasal cavity 101 and a lacrimal sac 102.

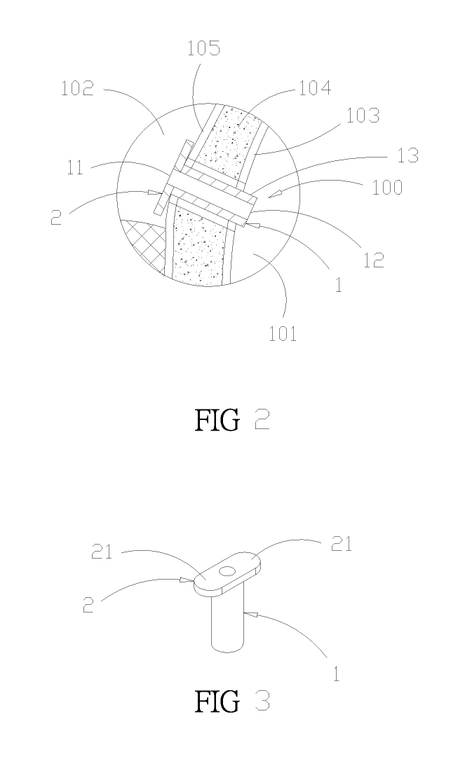

As shown in FIG. 2, the duct 100 for tear flow comprises: a tube 1 having a front end 11 and a rear end 12, wherein the tube is set across the a nasal mucosa 103, a nasal bone wall 104 and a lacrimal sac mucosa 105. Thereby the nasal cavity and lacrimal sac are connected; a positioning element 2, set on the front end of the tube and fastened on the lacrimal sac mucosa; so that tears flowing into the lacrimal sac can flow into the nasal cavity due to a connection between nasal cavity.

The Tube 1 is made of Titanium alloy material, set across a nasal mucosa 103, a nasal bone wall 104 and a lacrimal sac mucosa 105, wherein the nasal cavity 101 and lacrimal sac are 102 connected. It is achieved, that tears flowing into the lacrimal sac can flow into the nasal cavity due to the connection between the nasal cavity and the lacrimal sac.

Inside the surface of the tube 1, is handled with e.g. polishing and further more coated with hydrophobic material, so that tears can easily flow into the nasal cavity 101 through the tube.

The front end of the tube 1 is coated with antihistamine. Thereby it is insured, that the tube will not be obstructed by the lacrimal sac mucosa 105.

The rear end 12 of the tube 1 protrudes from the nasal mucosa 103. Thereby an anti-proliferative part 13 exists. It is insured, that the rear end of the tube will not be obstructed by the nasal mucosa. Besides, antihistamine is coated on surface of the said anti-proliferative part 13. Thereby it is further insured, that the rear end of the tube will not be obstructed by the lacrimal sac mucosa; meanwhile, the protrusion of the rear end of the tube is reduced. Thereby discomfort due to the protrusion of the rear end of the tube is avoided.

A flange can be set on the rear end 12 of the said tube 1 (not shown), wherein the flange is fastened outside the nasal mucosa. Thereby antihistamine coating on the rear end 12 of the tube 1 is reduced or saved, or the protrusion on the rear end of the tube is reduced.

As shown in FIG. 2˜3, the positioning element 2 set on the front end 11 of the tube 1, has at least two fastening parts 21, protruding from an outer side of the tube, wherein the fastening parts 21 are made of elastic material, e.g. plastic, are at a right angle to the tube. These fastening parts can alone with the front end of the tube easily cross through the nasal mucosa 103, the bone wall 104 and the lacrimal sac mucosa 105, and be easily fastened on the lacrimal sac mucosa.

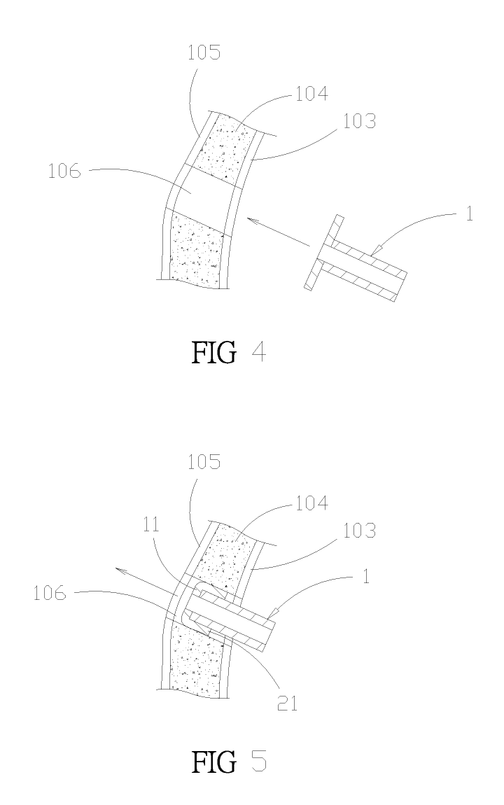

FIG. 4˜6 show a flow chart of installation of the tube 1. As shown FIG. 4, before installation of the tube 1 a passage 106 passing through the nasal mucosa 103, the bone wall 104 and the lacrimal sac mucosa 105 is opened, wherein the front end of the said passage is set as close as possible on the top of the obstruction of the duct for tear flow (as FIG. 1 shown), so that accumulation of tears is reduced. Besides, the front end of the passage is higher than the rear end, so that tears in the lacrimal sac can flow into the tube, then downwards into the nasal cavity.

As shown in FIG. 5, while the front end 11 of the tube 1 passes the passage 106 passing through the nasal mucosa 103, the bone wall 104 and the lacrimal sac mucosa 105, the fastening parts 21 are due to elasticity bent and attached on the tube. Thereby the fastening parts can alone with the front of the tube easily pass through the nasal mucosa, the bone wall and the lacrimal sac mucosa.

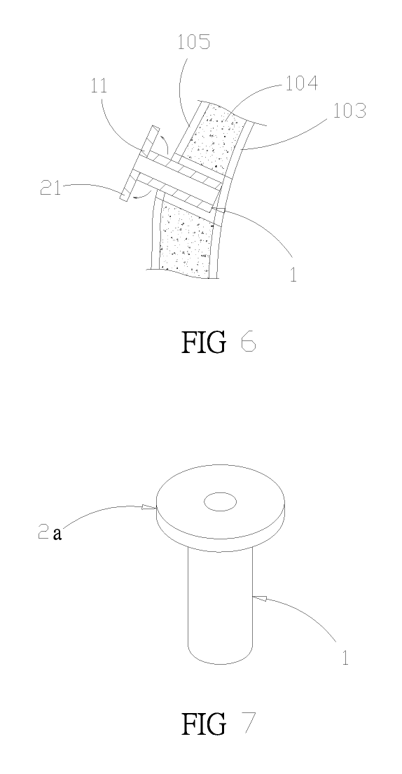

As shown in FIG. 6, the front end 11 of tube 1 slightly extends beyond the lacrimal sac 105, hence the fastening parts 21 become oriented at a right angle to the tube, then the tube is pulled back. Thereby the fastening parts are fastened on the lacrimal sac mucosa. It is insured, that the tube cannot be detached (as shown in FIG. 1˜2).

The two fastening parts 21 are set parallel to the tube (not shown). After crossing through the lacrimal sac mucosa the fastening parts are bent to protrude from the outer side of the tube at a right angle to the tube. Thereby the fastening parts are fastened on the lacrimal sac mucosa.

In the second embodiment as shown in FIG. 7, the positioning element 2a is a wafer body made of elastic material. Thereby alone with the front end of the tube the positioning element can easily cross through the nasal mucosa, the bone wall and the lacrimal sac mucosa, and be fastened on the lacrimal sac mucosa. Hence it is insured that the front end of the tube 1 cannot be obstructed by the lacrimal sac mucosa. That is, coating of antihistamine on the front end of the tube 1 is reduced or saved due to the positioning element 2a.

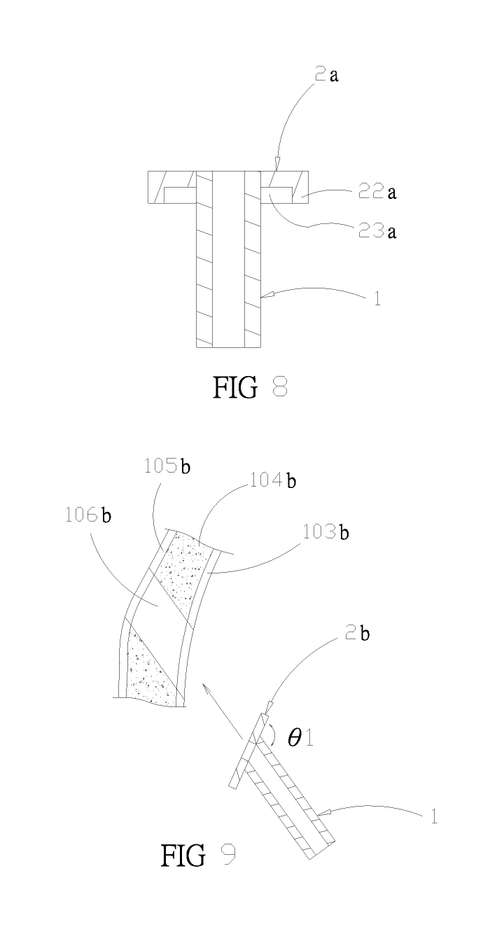

In the third embodiment as shown in FIG. 8, the flange of the positioning element 2a is furthermore set on an edge wall 22a extending downwards. Thereby a space 23a is created for growing and filling of lacrimal sac mucosa, hence stability of the tube is achieved.

In the fourth embodiment as shown in FIG. 9, since the front end of the passage 106b passing through the nasal mucosa 103b, the bone wall 104b and the lacrimal sac mucosa 105b is higher than the rear end, therefore the passage 106b is not in right angle to the lacrimal sac mucosa. In this case, there is a relevant, variant angle θ1 between the positioning element 2b and the tube 1, so that the positioning element 2b is attached to the lacrimal sac mucosa as flatly as possible.

In the embodiments above, the tube 1 and the positioning element are made of different materials. Of course, a uniform material and manufacturing as one piece are possible, as well.

1. A duct for tear flow applied for connecting a nasal cavity and a lacrimal sac, comprises:
a tube, having a front end and a rear end, wherein, the said tube is set across the the nasal mucosa, the bone wall and the lacrimal sac mucosa, so that the nasal cavity and the lacrimal sac are connected;
a positioning element, set on the front end of the said tube, fastened on the lacrimal sac mucosa.

2. A duct for tear flow of claim 1 wherein on surface of the front end of the said tube is coated with antihistamine.

3. A duct for tear flow of claim 1, wherein the rear end of the tube extends beyond the nasal mucosa, so that an anti-proliferative part exists.

3. for tear flow of claim 3, wherein on surface of the said anti-proliferative part is coated with antihistamine.

5. A duct for tear flow of claim 1, wherein the positioning element has at least two fastening parts protruded outside of the tube.

6. A duct for tear flow of claim 5, wherein said two fastening parts are oriented at a right angle to the tube and are made of elastic material.

7. A duct for tear flow of claim 5, wherein the two fastening parts are oriented parallel to the tube, allowing to be bent outwards of the tube at a right angle to the tube.

8. A duct for tear flow of claim 1, wherein the positioning element is a wafer body made of elastic material.

9. A duct for tear flow of claim 8, wherein the flange of the positioning element is set on an edge wall extending downwards, so that a space is formed.

10. A duct for tear flow of claim 1, wherein on the rear end of the tube there is a flange fastened outer the nasal mucosa.
